(19) United States
(12) Patent Application Publication
(10) Pub. No.: US 2022/0202971 A1
(43) Pub. Date: Jun. 30, 2022

(54) STERILIZATION DEVICE

(71) Applicant: Enplas Corporation, Saitama (JP)

(72) Inventors: Toshinori OKADA, Saitama (JP); Tetsuya TOKIWA, Saitama (JP); Natsuki INAOKA, Saitama (JP)

(73) Assignee: Enplas Corporation, Saitama (JP)

(21) Appl. No.: 17/557,075

(22) Filed: Dec. 21, 2021

(30) Foreign Application Priority Data
Dec. 28, 2020 (JP) 2020-218782

(51) Int. Cl. A61L 2/10

(52) U.S. Cl. CPC A61L 2/10; A61L 2202/20; A61L 2202/10

(57) ABSTRACT

A sterilization device includes: a reservoir, a supply port, an output port and a light source. The reservoir part includes a first half reservoir part having a substantially hemispherical shape and located on an upstream side in a flow direction of the fluid at the supply port, and a second half reservoir part having a substantially hemispherical shape and located on a downstream side in the flow direction. The supply port opens to the first half reservoir part. The output port opens to the second half reservoir part. When the supply port and the output port are projected on a virtual plane orthogonal to an extending direction of an inner surface of a supply channel connected to the reservoir part at the supply port, a center of gravity of the supply port and a center of gravity of the output port are separated from each other.

### CROSS REFERENCE TO RELATED APPLICATIONS

This application is entitled to the benefit of Japanese Patent Application No. 2020-218782, filed on Dec. 28, 2020, the disclosure of which including the specification, drawings and abstract is incorporated herein by reference in its entirety.

### TECHNICAL FIELD

The present invention relates to a sterilization device that sterilizes a fluid by irradiating the fluid with an ultraviolet ray.

### BACKGROUND ART

It is widely known that fluid such as liquid can be sterilized by using an ultraviolet ray. For example, PTL 1 discloses a fluid sterilization device that sterilizes fluid flowing through a channel by irradiating a channel extended in an axial direction along the axial direction with an ultraviolet ray.

To be more specific, the fluid sterilization device disclosed in PTL 1 includes a light source including a semiconductor light-emitting element that emits an ultraviolet ray, and a housing including a channel through which the fluid to be sterilized flows in the axial direction. The light source is disposed in the housing at one end portion in the axis direction. The housing has a tapered structure in which the cross-sectional area of the channel gradually increases from one end portion toward the other end portion. The tapered structure has an inclination that matches the orientation angle of the semiconductor light-emitting element.

In addition, a rectification means that regulates the flow of the fluid is disposed at the other end portion of the housing.

With the housing having the taper structure with the inclination that matches the orientation angle of the semiconductor light-emitting element, the fluid sterilization device disclosed in PTL 1 can deliver an ultraviolet ray to a position remote from the light source. In addition, in the fluid sterilization device disclosed in PTL 1, the fluid whose flow has been regulated by the rectification means is irradiated with an ultraviolet ray so as to evenly irradiate the fluid with the ultraviolet ray, and thus the sterilization effect can be can increased.

### CITATION LIST

### Patent Literature

PTL 1

Japanese Patent Application Laid-Open No. 2019-98055

### SUMMARY OF INVENTION

### Technical Problem

The sterilization device disclosed in PTL 1 has a room for improvement from the viewpoint of uniformly irradiating the fluid with an ultraviolet ray, and from the viewpoint of reducing the pressure drop of the fluid.

In view of this, an object of the present invention is to provide a sterilization device that can sufficiently sterilize fluid by uniformly irradiating the fluid with an ultraviolet ray.

### Solution to Problem

A sterilization device according to an embodiment of the present invention is configured to sterilize fluid by irradiating the fluid with an ultraviolet ray, the sterilization device including: a reservoir part having a substantially spherical shape and configured to store the fluid; a supply port configured to be open to the reservoir part and configured to supply the fluid into the reservoir part; an output port configured to be open to the reservoir part and configured to output the fluid in the reservoir part; and a light source configured to emit an ultraviolet ray into the reservoir part. The reservoir part includes a first half reservoir part having a substantially hemispherical shape and located on an upstream side in a flow direction of the fluid at the supply port, and a second half reservoir part having a substantially hemispherical shape and located on a downstream side in the flow direction. The supply port opens to the first half reservoir part. The output port opens to the second half reservoir part. When the supply port and the output port are projected on a virtual plane orthogonal to an extending direction of an inner surface of a supply channel connected to the reservoir part at the supply port, a center of gravity of the supply port and a center of gravity of the output port are separated from each other.

### Advantageous Effects of Invention

According to the present invention, it is possible to provide a sterilization device that can sufficiently sterilize fluid by uniformly irradiating the fluid with an ultraviolet ray.

### BRIEF DESCRIPTION OF DRAWINGS

FIG. 1 is a perspective view of a sterilization device according to an embodiment;

FIG. 2 is a cross-sectional perspective view of the sterilization device according to the embodiment;

FIG. 3 is a see-through perspective view of the sterilization device according to the embodiment;

FIG. 4 is a projection diagram illustrating a positional relationship between a supply port and an output port when the supply port, the output port and a reservoir part are projected on a virtual plane; and

FIG. 5 is a diagram illustrating a flow of fluid in a reservoir part of the sterilization device according to the embodiment.

### DESCRIPTION OF EMBODIMENTS

A sterilization device according to an embodiment of the present invention is described below.

### Configuration of Sterilization Device

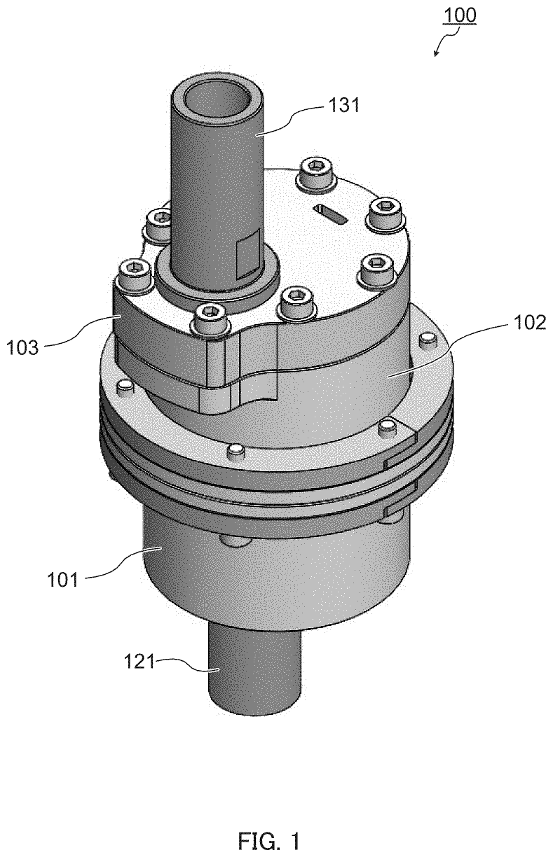

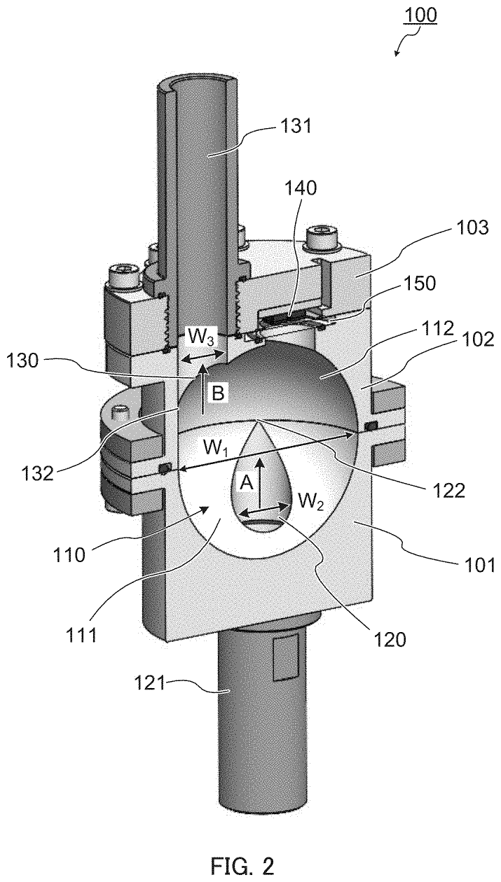

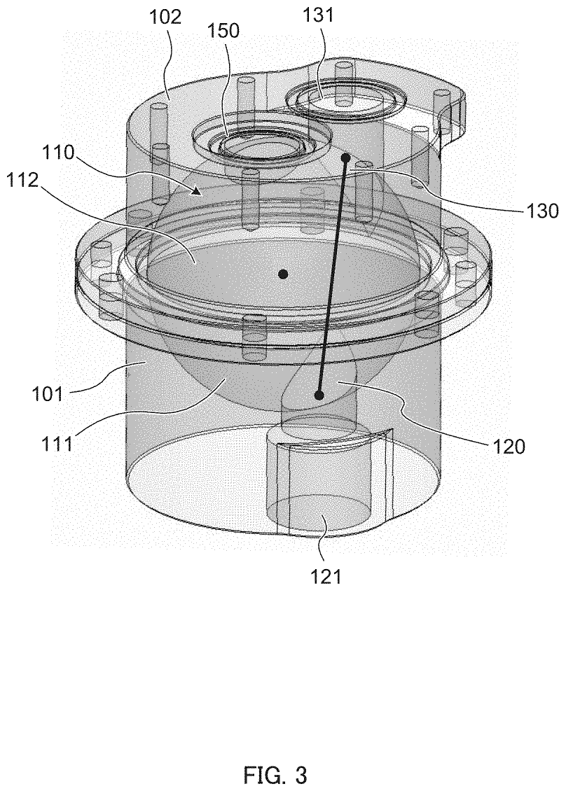

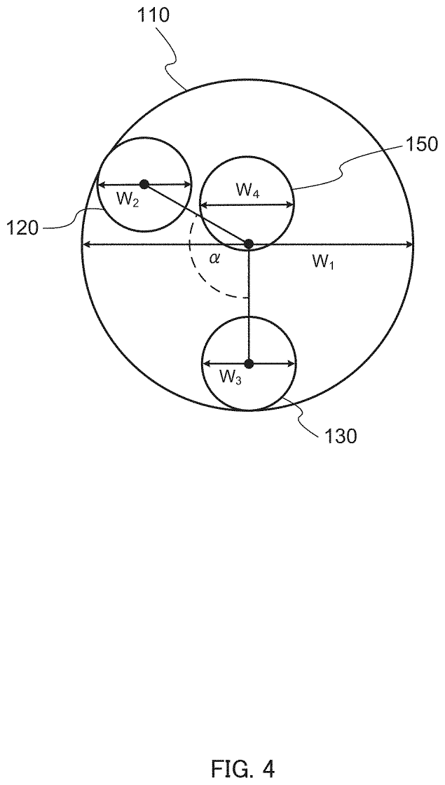

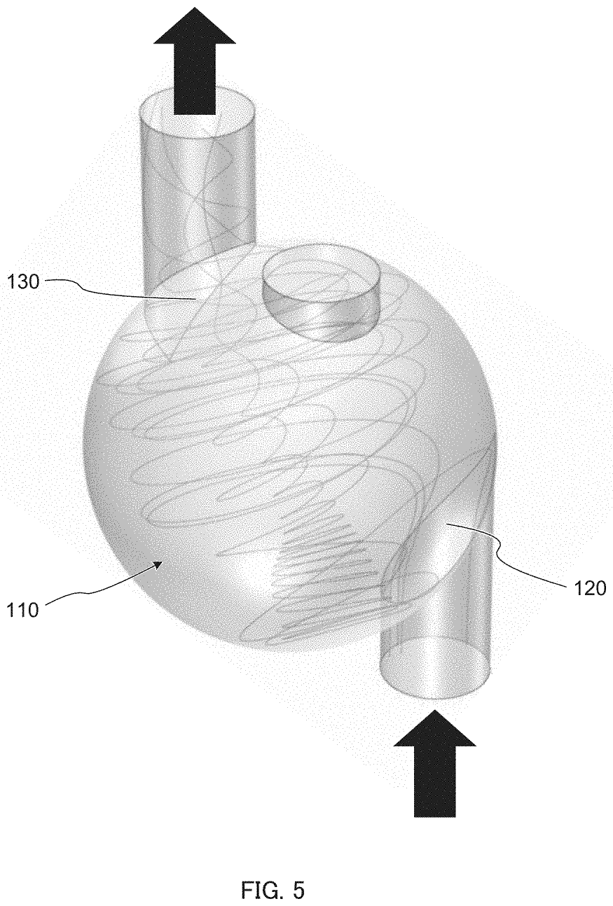

FIGS. 1 to 3 illustrate a configuration of sterilization device 100 according to the embodiment of the present invention. FIG. 1 is a perspective view of sterilization device 100. FIG. 2 is a cross-sectional perspective view of sterilization device 100. FIG. 3 is a see-through perspective view of a region around reservoir part 110 of sterilization device 100. FIG. 4 is a projection diagram illustrating a positional relationship between supply port 120 and output port 130 when supply port 120, output port 130 and reservoir part 110 are projected on a virtual plane (described later). FIG. 5 is a diagram illustrating a flow of fluid in reservoir part 110 of sterilization device 100 according to the embodiment.

As illustrated in FIGS. 1 and 2, sterilization device 100 includes reservoir part 110 for storing the fluid to be sterilized, supply port 120 that opens to reservoir part 110, output port 130 that opens to reservoir part 110, and light source 140 that emits an ultraviolet ray into reservoir part 110. Sterilization device 100 according to the present embodiment is an apparatus that sterilizes fluid by irradiating the fluid with an ultraviolet ray.

Reservoir part 110 is a substantially spherical space for storing fluid. Reservoir part 110 includes substantially hemispherical first half reservoir part 111 located on the upstream side and substantially hemispherical second half reservoir part 112 located on the downstream side in the flow direction (the arrow A direction in FIG. 2) of the fluid at supply port 120. In the present embodiment, reservoir part 110 is formed by joining first member 101 including first half reservoir part 111 and second member 102 including second half reservoir part 112. In the present embodiment, the flange of first member 101 and the flange of second member 102 are joined by using a plurality of screws. Note that third member 103 may be further joined to second member 102. Second member 102 and third member 103 are joined by using a plurality of screws.

The wall around reservoir part 110 is configured to be not deformed or damaged by the pressure of the flowing fluid. The wall around reservoir part 110 is composed of metal or resin, for example. In addition, from the viewpoint of efficiently emitting an ultraviolet ray to the fluid in reservoir part 110, it is preferable that the wall (inner surface) around reservoir part 110 (first half reservoir part 111 and second half reservoir part 112) include an ultraviolet ray reflection surface whose reflectance to the ultraviolet ray emitted from light source 140 is 80% or greater. The ultraviolet ray reflection surface can be provided at the inner surface of reservoir part 110 by forming the inner surface of reservoir part 110 with a material with high reflectance to ultraviolet rays such as polytetrafluoroethylene (PTFE) and aluminum, for example. In the present embodiment, both first member 101 and second member 102 are composed of PTFE.

Internal diameter W1 of reservoir part 110 is not limited, but is, for example, approximately 20 to 60 mm. When internal diameter W1 of reservoir part 110 is approximately 20 to 60 mm, the fluid in reservoir part 110 can be sufficiently sterilized even when only one UV-C LED is used as light source 140.

Supply port 120 is an opening that opens to reservoir part 110, for supplying fluid into reservoir part 110. Output port 130 is an opening that open to reservoir part 110, for outputting the sterilized fluid in reservoir part 110. Supply port 120 opens to first half reservoir part 111 in reservoir part 110, and output port 130 opens to second half reservoir part 112 in reservoir part 110. Preferably, output port 130 is disposed at a position where the ultraviolet ray emitted from light source 140 does not directly reaches in reservoir part 110 (second half reservoir part 112). In the present embodiment, supply port 120 is connected to supply channel 121, and output port 130 is connected to output channel 131. In addition, in the present embodiment, a portion of supply channel 121 in the vicinity of supply port 120 and a portion of output channel 131 in the vicinity of output port 130 are parallel to each other.

In the present embodiment, supply port 120 is connected to supply channel 121. In other words, the opening of supply channel 121 to reservoir part 110 is supply port 120. Preferably, supply port 120 (supply channel 121) is disposed such that the fluid can be smoothly supplied into reservoir part 110 along the wall of reservoir part 110. In the present embodiment, at connecting part 122, a part of the inner surface of supply channel 121 is disposed such that it is smoothly contiguous with the inner surface of reservoir part 110 and coincides with the tangent to the inner surface of reservoir part 110 at connecting part 122. Here, connecting part 122 is the contact point of the inner surface of supply channel 121 and the inner surface of reservoir part 110 in a cross section taken along the flow direction of the fluid at supply port 120 (arrow A direction in FIG. 2) and including the center of gravity of reservoir part 110.

In addition, in the present embodiment, output port 130 is connected to output channel 131. In other words, the opening of output channel 131 to reservoir part 110 is output port 130. Preferably, output port 130 (output channel 131) is disposed such that the fluid can be smoothly output from the inside of reservoir part 110 along the wall of reservoir part 110. In the present embodiment, at connecting part 132, a part of the inner surface of output channel 131 is disposed such that it is smoothly contiguous with the inner surface of reservoir part 110 and coincides with the tangent to the inner surface of reservoir part 110 at connecting part 132. Here, connecting part 132 is the contact point of the inner surface of output channel 131 and the inner surface of reservoir part 110 in a cross section taken along the flow direction (arrow B direction in FIG. 2) of the fluid at output port 130 and including the center of gravity of reservoir part 110.

By eliminating steps at connecting part 122 of the inner surface of supply channel 121 and the inner surface of reservoir part 110 and connecting part 132 of the inner surface of output channel 131 and the inner surface of reservoir part 110, a fluid flow along the surface of the wall of spherical reservoir part 110 can be provided, and the fluid can be output after keeping the fluid by rotating it in a specific direction in reservoir part 110. In this manner, the fluid is uniformly irradiated with the ultraviolet ray, and the fluid can be sufficiently sterilized.

Internal diameter W2 of supply port 120 (supply channel 121) and internal diameter W3 of output port 130 (output channel 131) are not limited. Preferably, internal diameter W2 of supply port 120 (supply channel 121) and internal diameter W3 of output port 130 (output channel 131) are within a range of 25 to 40% of internal diameter W1 of reservoir part 110 from the viewpoint of reducing the pressure drop of the fluid while maintaining the sterilization performance. With internal diameter W2 of supply port 120 and internal diameter W3 of output port 130 having large sizes, the pressure drop of the fluid at sterilization device 100 can be reduced. On the other hand, with internal diameter W2 of supply port 120 and internal diameter W3 of output port 130 having small sizes, the time during which the fluid supplied from supply port 120 is kept at reservoir part 110 is lengthened, and the sterilization performance can be improved.

As illustrated in FIG. 4, supply port 120 and output port 130 are disposed such that when supply port 120, output port 130 and reservoir part 110 are projected on a virtual plane orthogonal to the extending direction (arrow A direction in FIG. 2) of the inner surface of supply channel 121 connected to reservoir part 110 at supply port 120, the center of gravity of supply port 120 and the center of gravity of output port 130 are separated from each other.

In the case where supply port 120 and output port 130 are disposed in the above-described manner, the fluid supplied from supply port 120 into reservoir part 110 reaches output port 130 after repeatedly rotating in reservoir part 110 without linearly going toward output port 130, as illustrated in FIG. 5. Thus, the fluid reaches output port 130 after being sufficiently sterilized by irradiation with a sufficient amount of ultraviolet ray. Note that in the present embodiment, window 150 (light source 140) is disposed such that it does not overlap supply port 120 and output port 130 when projected as described above. Note that in the present embodiment, as illustrated in FIG. 4, supply port 120, output port 130, window 150 and reservoir part 110 are circles when projected on the above-mentioned virtual plane. Therefore, the center of gravity of supply port 120 coincides with the center of supply port 120, the center of gravity of output port 130 coincides with the center of output port 130, and the center of gravity of reservoir part 110 coincides with the center of reservoir part 110.

In the present embodiment, as illustrated in FIG. 3, the center of gravity of reservoir part 110 (the black point at the center of reservoir part 110 in FIG. 3) is separated from the straight line connecting the center of gravity of supply port 120 (the black point at the center of supply port 120 in FIG. 3) and the center of gravity of output port 130 (the black point at the center of output port 130 in FIG. 3). In addition, the center of gravity of reservoir part 110 is not located between supply port 120 and output port 130.

In addition, in the present embodiment, as illustrated in FIG. 4, it is preferable that when supply port 120, output port 130 and reservoir part 110 are projected on the above-mentioned virtual plane, angle α between the straight line connecting the center of gravity of supply port 120 (the black point at the center of supply port 120 in FIG. 4) and the center of gravity of reservoir part 110 (the black point at the center of reservoir part 110 in FIG. 4), and the straight line connecting the center of gravity of output port 130 (the black point at the center of output port 130 in FIG. 4) and the center of gravity of reservoir part 110 be within a range of 75 to 165°, more preferably within a range of 120 to 150°. With angle α within the above-mentioned within range, the pressure drop of the fluid can be reduced while maintaining the sufficient sterilization performance. Here, an angle between two straight lines means the smaller angle of the two angles formed by the two straight lines.

Light source 140 irradiates the fluid in reservoir part 110 with an ultraviolet ray. Light source 140 may directly irradiate the fluid in reservoir part 110 with an ultraviolet ray, or may irradiate the fluid in reservoir part 110 with an ultraviolet ray through other members such as a window and a mirror. In the present embodiment, the wall forming reservoir part 110 includes window 150 that transmits an ultraviolet ray, and light source 140 emits an ultraviolet ray into reservoir part 110 through window 150. The type of light source 140 is not limited as long as an ultraviolet ray can be emitted. Examples of light source 140 include a light-emitting diode (LED), a mercury lamp, a metal halide lamp, a xenon lamp, and a laser diode (LD). In the present embodiment, light source 140 is a light-emitting diode (LED). The wavelength of the ultraviolet ray emitted by light source 140 is not limited. Preferably, the wavelength of the ultraviolet ray emitted by light source 140 is 200 nm to 350 nm, more preferably 200 nm to 280 nm from the viewpoint of effectively sterilizing the fluid in reservoir part 110. Specifically, preferably, the ultraviolet ray emitted from light source 140 is ultraviolet C (UVC). Examples of commercially available light source 140 include NCSU334A (NICHIA CORPORATION), which is an ultraviolet ray light-emitting diode whose peak wavelength is 280 nm. In addition, other examples of the ultraviolet ray light-emitting diode whose peak wavelength is 280 nm include KLARAN (ASAHI KASEI CORPORATION) and ZEU110BEAE (STANLEY ELECTRIC CO., LTD).

The position of light source 140 is not limited as long as the fluid in reservoir part 110 can be irradiated with an ultraviolet ray, but the sterilization performance can be improved when it is disposed at second half reservoir part 112. In the present embodiment, light source 140 is disposed on the second half reservoir part 112 side. To be more specific, light source 140 is disposed inside the recess provided in third member 103 (inside the wall making up second half reservoir part 112) such that the optical axis of light source 140 does not intersect supply port 120 or output port 130.

Window 150 is disposed as a part of the wall surface of reservoir part 110, and transmits the ultraviolet ray emitted from light source 140, to the inside of reservoir part 110. The material of window 150 is not limited as long as it can transmit an ultraviolet ray and has required strength. Preferably, the material of window 150 is a material that transmits an ultraviolet ray whose wavelength is 200 nm to 350 nm, or more preferably a material that transmits an ultraviolet ray of 200 nm to 280 nm from the viewpoint of improving the sterilization performance. Examples of the material of window 150 include quartz (SiO₂), sapphire (Al₂O₃) and amorphous fluorine resin.

In addition, the shape of window 150 is not limited as long as the ultraviolet ray emitted from light source 140 can reach the inside of reservoir part 110, and the shape may be a plate shape, or a shape that matches the inner surface of reservoir part 110. In the present embodiment, window 150 has a plate shape, and is disposed inside a recess provided in second member 102. Outer diameter W4 of window 150 is not limited as long as the ultraviolet ray emitted from light source 140 can reach the inside of reservoir part 110. For example, preferably, the size of outer diameter W4 of window 150 is 20 to 50% of the size of internal diameter W1 of reservoir part 110. With window 150 having a large outer diameter W4, a wide range in reservoir part 110 can be directly irradiated with the ultraviolet ray. On the other hand, with window 150 having a small outer diameter W4, the ratio of the size of the ultraviolet ray reflection surface with respect to the inner surface of reservoir part 110 can be increased.

### Usage of Sterilization Device

Next, a usage of sterilization device 100 according to the present embodiment is described.

In the state where an ultraviolet ray is emitted from light source 140, the fluid to be sterilized (for example water) is introduced from supply port 120 into reservoir part 110, and the fluid in reservoir part 110 is taken out from output port 130. At this time, the fluid may be moved by applying a pressure on supply port 120 (supply channel 121) side, or the fluid may be moved by depressurizing the output port 130 (output channel 131) side. As described above, in sterilization device 100 according to the present embodiment, reservoir part 110 has a substantially spherical shape, and supply port 120 and output port 130 are disposed to meet a predetermined condition. Thus, the fluid to be sterilized is irradiated with an ultraviolet ray while being rotated in reservoir part 110, and is output from output port 130 in a sufficiently sterilized state.

### Effect

As described above, in sterilization device 100 according to the present embodiment, reservoir part 110 has a substantially spherical shape, and supply port 120 and output port 130 are disposed to meet a predetermined condition. It is thus possible to reduce the pressure drop of the fluid while maintaining the sufficient sterilization performance.

### INDUSTRIAL APPLICABILITY

The sterilization device according to the present embodiment is useful for sterilization of clean water, agricultural water, food washing water, various types of washing water, bathing water, and pool water, for example.

### REFERENCE SIGNS LIST

100 Sterilization device
101 First member
102 Second member
103 Third member
110 Reservoir part
111 First half reservoir part
112 Second half reservoir part
120 Supply port
121 Supply channel
122 Connecting part
130 Output port
131 Output channel
132 Connecting part
140 Light source
150 window

1. A sterilization device configured to sterilize fluid by irradiating the fluid with an ultraviolet ray, the sterilization device comprising:
a reservoir part having a substantially spherical shape and configured to store the fluid;
a supply port configured to be open to the reservoir part and configured to supply the fluid into the reservoir part;
an output port configured to be open to the reservoir part and configured to output the fluid in the reservoir part; and
a light source configured to emit an ultraviolet ray into the reservoir part,
wherein the reservoir part includes a first half reservoir part having a substantially hemispherical shape and located on an upstream side in a flow direction of the fluid at the supply port, and a second half reservoir part having a substantially hemispherical shape and located on a downstream side in the flow direction,
wherein the supply port opens to the first half reservoir part,
wherein the output port opens to the second half reservoir part, and
wherein when the supply port and the output port are projected on a virtual plane orthogonal to an extending direction of an inner surface of a supply channel connected to the reservoir part at the supply port, a center of gravity of the supply port and a center of gravity of the output port are separated from each other.

2. The sterilization device according to claim 1, wherein when the supply port and the output port are projected on the virtual plane, the supply port and the output port are separated from each other.

3. The sterilization device according to claim 1, wherein the light source is disposed on the second half reservoir part side.

4. The sterilization device according to claim 1, wherein a center of gravity of the reservoir part is separated from a straight line connecting the center of gravity of the supply port and the center of gravity of the output port.

5. The sterilization device according to claim 4, wherein the center of gravity of the reservoir part is located between the supply port and the output port.

6. The sterilization device according to claim 1, wherein when the supply port, the output port and the reservoir part are projected on the virtual plane, an angle between a straight line connecting the center of gravity of the supply port and a center of gravity of the reservoir part and a straight line connecting the center of gravity of the output port and the center of gravity of the reservoir part is within a range of 75 to 165°.

7. The sterilization device according to claim 1,
wherein the supply port is connected to the supply channel; and
wherein at a connecting part of the inner surface of the supply channel and an inner surface of the reservoir part in a cross section taken along the flow direction of the fluid at the supply port and including a center of gravity of the reservoir part, a part of the inner surface of the supply channel is disposed such that the part of the inner surface of the supply channel is smoothly contiguous with the inner surface of the reservoir part and coincides with a tangent to the inner surface of the reservoir part at the connecting part.

8. The sterilization device according to claim 1, wherein the output port is disposed at a position where an ultraviolet ray emitted from the light source does not directly reaches.

9. The sterilization device according to claim 1,
wherein a wall making up the reservoir part includes a window configured to transmit an ultraviolet ray; and
wherein the light source emits an ultraviolet ray into the reservoir part through the window.

10. The sterilization device according to claim 1, wherein an inner surface of the reservoir part includes an ultraviolet ray reflection surface whose reflectivity to an ultraviolet ray is 80% or greater.
